(19) United States
(12) Patent Application Publication
(10) Pub. No.: US 2011/0021484 A1
(43) Pub. Date: Jan. 27, 2011

(54) Gallium compounds and methods of use to treat inflammatory bowel disease

(76) Inventor: Lawrence Richard Bernstein, Menlo Park, CA (US)

Correspondence Address: Lawrence R. Bernstein, 285 Willow Road, Menlo Park, CA, 94025-2711, US

(21) Appl. No.: 12/920,798

(22) PCT Filed: Mar. 6, 2009
(86) PCT No.: PCT/US09/36279

§ 371 Date: Sep. 2, 2010

Related U.S. Application Data
(60) Provisional application No. 61/034,889, filed on Mar. 7, 2008.

(51) Int. Cl. A61K 31/555; C07F 5/00; A61P 1/00

(52) U.S. Cl. 514/184; 549/210

(57) ABSTRACT

Provided are formulations and methods for treating inflammatory bowel disease, including Crohn's disease, ulcerative colitis, and Johne's disease. The formulations provided comprise pharmaceutically acceptable gallium compounds; the methods provided comprise means of using such formulations in the treatment of inflammatory bowel disease and closely related disorders.

### TECHNICAL FIELD

This invention relates generally to treatments for inflammatory bowel disease. In particular, this invention pertains to pharmaceutical formulations comprising gallium and their uses in treating Crohn's disease, ulcerative colitis, and other types of inflammatory bowel disease.

### BACKGROUND OF THE INVENTION

The most prevalent forms of inflammatory bowel disease (IBD) are Crohn's disease (CD) and ulcerative colitis (UC). Other diseases and disorders classified under the category of IBD include Behçet's syndrome, collagenous colitis, diversion colitis, indeterminate colitis, infective colitis, ischemic colitis, and lymphocytic colitis.

Crohn's disease and ulcerative colitis are most prevalent in developed countries, particularly in North America and Europe, where the combined prevalence is about 100 to 200 cases per 100,000 population (Cho J H, Inflammatory bowel disease: Genetic and epidemiologic considerations. World J Gastroenterol 2008; 14(3): 338-347). The prevalence in less-developed countries is commonly one or two orders of magnitude lower. This discrepancy has been ascribed to the “hygiene hypothesis”: exposure to an abundance of microbes and parasites early in life (as occurs more commonly in less-developed countries) can program the immune system to correctly identify pathogens and ignore self tissues and harmless substances, whereas a lack of such exposure (as occurs commonly in the more hygienic environments of developed countries) may result (at least in genetically predisposed individuals) in the immune system pathologically reacting to self tissues and/or harmless environmental substances.

Crohn's disease is characterized by granulomatous inflammation of portions of the gastrointestinal tract. The inflammation is most common in the small intestine and colon; inflammation of the rectum is less common, while inflammation of the mouth, tongue, esophagus, stomach, and duodenum are uncommon. Only some segments of the gastrointestinal tract are typically affected, with other segments (“skip zones”) remaining healthy. Crohn's disease usually affects all histological layers of the bowel. Individuals with CD have recurrent bouts of symptoms, often severe, that can include abdominal pain, diarrhea, nausea, anorexia, weight loss, fever, fatigue, and perianal fistulas and abscesses.

In ulcerative colitis, inflammation begins in the rectum and from there it may spread up the colon. There is usually a sharp demarcation between affected and healthy tissue, and there are no “skip zones” as occur in CD. Typically only the mucosal and submucosal layers of the bowel are affected. Symptoms of UC commonly include diarrhea, which is frequently bloody, abdominal pain and cramps, fever, weight loss, and sweats.

Both CD and UC are sometimes accompanied by non-gastrointestinal inflammation and other disorders. Such manifestations include uveitis, iritis, episcleritis, arthritis, enthesitis, skin disorders, thrombosis, osteoporosis, neurological disease, and liver disease.

While most cases of IBD do not directly cause death, mortality is raised by IBD due to an increase in the incidence of sepsis associated with perforation and ensuing peritonitis, cancer, thromboembolic disease, severe malnutrition, and complications from surgery or drug treatment.

The causes of CD and UC, as well as other inflammatory bowel diseases, are not well understood, but are thought to involve immunological, genetic, and infectious components. Based on current knowledge, it appears that individuals genetically predisposed towards IBD are susceptible to pathological immunological reactions in the gastrointestinal tract. These reactions may be triggered by infectious agents, normal intestinal flora, or perhaps certain ingested foods. Immunological reactions initiated by these triggers may persist due to continued exposure to them, or due to ongoing autoimmune reactions. T-cell involvement is very common, if not ubiquitous. One hypothesis regarding CD implicates infection with Mycobacterium as causative, at least in some cases. There is substantial experimental backing for this hypothesis, including the presence of a nearly identical disease in cattle, Johne's disease, which is caused by Mycobacterium avium subspecies paratuberculosis.

Current treatments for inflammatory bowel disease include non-steroidal anti-inflammatory agents (NSAIDS) such as sulfasalzine, mesalazine (5-aminosalicylic acid), balsalazide, and olsalazine; anti-inflammatory corticosteroids such as prednisone, budesonide, methylprednisolone, and hydrocortisone; the anti-inflammatory cytokine interferon-beta-1a; immunosuppressants such as azathioprine, mercaptopurine, and cyclosporine; antibiotics such as metronidazole and ciprofloxacin; tumor necrosis factor alpha (TNFα) inhibitors such as infliximab, etanercept, and adalimumab; the anti-integrin natalizumab; the antimetabolite and antifolate methotrexate; and, in severe cases, surgery to remove sections of damaged bowel. Experimental helminthic therapy has shown some success, which supports the validity of the hygiene hypothesis, mentioned above.

Gallium compounds, including gallium nitrate and gallium maltolate, have been repeatedly shown to have antimicrobial activities in vitro and in vivo, particularly against intracellular organisms (Olakanmi O et al., Infection and Immunity 68:5619-5627, 2000; Harrington J R et al., Journal of Veterinary Pharmacology and Therapeutics 29:121-127, 2006; United States Patent Applications 2006/0222628, 2003/0083308, and 2002/0068761). Activity has been shown against many pathological intracellular microorganisms, including intracellular bacteria such as Mycobacterium, and Rhodococcus; intracellular protists such as Plasmodium and trypanosomes; viruses such as HIV and herpes viruses; and others. Gallium has also shown activity against many extracellular organisms. These include, among other species, Pseudomonas aeruginosa (highly potent activity, including against biofilms; e.g., DeLeon K et al., Antimicrobial Agents and Chemotherapy, doi:10.1128/AAC.01330-08, 2009; Kaneko Y et al., Journal of Clinical Investigation 117(4): 877-888, 2007; U.S. Pat. No. 5,997,912); spirochete bacteria and trypanosome parasites (Levaditi C et al., Le gallium, propriétés thérapeutiques dans la syphilis et les trypanosomiases expérimentales, C R Hebd Seances Acad Sci Ser D Sci Nat 192:1142-1143, 1931); the fungus Neurospora crassa (Winkelman G et al., Archiv fur Mikrobiologie 92:285-300 (1973); and the bacterium Escherichia coli (Hubbard J A M et al., Archives of Microbiology 146:80-86, 1986). Therapeutic efficacy for gallium has also been reported in animal models of inflammatory and autoimmune diseases, such as rheumatoid arthritis (Delbarre F, Rabaud M, Comptes Rendus de l'Académie des Sciences, Series D 283:1469-1472, 1976; Matkovic Vet al., Current Therapeutic Research 50:255-267, 1991; U.S. Pat. No. 5,175,006; U.S. Patent Application 2005/0220895), multiple sclerosis (Whitacre C et al., Journal of Neuroimmunology 39:175-182, 1992), uveitis (Lobanoff M C et al., Experimental Eye Research 65:797-801, 1997), and Type 1 diabetes (Flynn J O et al., Diabetes 41:38A, 1992). Systemically administered gallium has also shown efficacy in the treatment of cancer (Bernstein L R, Pharmacol Rev 50:665-682, 1998). Locally administered gallium is effective in treating psoriasis and related dermatologic disorders (U.S. Pat. No. 5,747,482).

It has now been discovered that gallium-containing compounds can potently treat inflammatory bowel disease. Pharmaceutical compositions of gallium and methods for treatment of inflammatory bowel disease are discussed herein.

### SUMMARY OF THE INVENTION

The present invention provides pharmaceutical formulations, methods, and drug delivery systems for treating inflammatory bowel disease.

In some embodiments of the present invention are provided methods for treating inflammatory bowel disease in an individual in need thereof, comprising administering to the individual a therapeutically effective amount of a gallium compound.

In some of these embodiments, the inflammatory bowel disease is Crohn's disease, ulcerative colitis, Johne's disease, Behçet's syndrome, collagenous colitis, diversion colitis, indeterminate colitis, infective colitis, ischaemic colitis, or lymphocytic colitis. In other embodiments, the inflammatory bowel disease is Crohn's disease, ulcerative colitis, or Johne's disease. In other embodiments, the inflammatory bowel disease is Crohn's disease. In other embodiments, the inflammatory bowel disease is ulcerative colitis. In other embodiments, the inflammatory bowel disease is Johne's disease.

In some embodiments, the gallium compound is selected from any pharmaceutically acceptable gallium compound. In other embodiments, the gallium compound is selected from the group consisting of gallium nitrate, gallium sulfate, gallium citrate, gallium chloride, gallium complexed with a 3-hydroxy-4-pyrone, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium (III)-gallium(III) tetrachloride, gallium pyridoxal isonicotinoyl hydrazone, gallium complexed with kenpaullone, and gallium complexed with a kenpaullone derivative. In other embodiments, the gallium compound is selected from the group consisting of gallium nitrate, gallium citrate, gallium chloride, gallium maltolate, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, and gallium 8-quinolinolate. In other embodiments, the gallium compound is selected from the group consisting of gallium nitrate, gallium citrate, gallium chloride, gallium complexed with a 3-hydroxy-4-pyrone, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium (III)-gallium(III) tetrachloride, gallium pyridoxal isonicotinoyl hydrazone, gallium complexed with kenpaullone, and gallium complexed with a kenpaullone derivative. In other embodiments, the gallium compound is a gallium complex of a 3-hydroxy-4-pyrone. In other embodiments, the gallium compound is gallium maltolate.

In some of these embodiments, the gallium compound is administered by any medically acceptable means. In some of these embodiments, the gallium compound is administered enterally. In other embodiments, the gallium compound is administered orally. In other embodiments, the gallium compound is administered rectally.

In some of these embodiments, the gallium compound is in a formulation further comprising a pharmaceutically acceptable carrier. In some of these embodiments, the formulation is in the form of a suppository. In other of these embodiments, the formulation is in the form of a tablet or a capsule. In other of these embodiments, the formulation is a liquid.

In some embodiments of the present invention is provided a pharmaceutically acceptable formulation comprising a gallium compound, the gallium from which is retained within the lumen and tissues of the gastrointestinal tract in an amount of at least about ten percent of the administered gallium dose for at least about twelve hours following administration. In other embodiments of the present invention is provided a pharmaceutically acceptable formulation comprising a gallium compound, the gallium from which is retained within the lumen and tissues of the gastrointestinal tract in an amount of at least about twenty-five percent of the administered gallium dose for at least about twelve hours following administration. In other embodiments of the present invention is provided a pharmaceutically acceptable formulation comprising a gallium compound, the gallium from which is retained within the lumen and tissues of the gastrointestinal tract in an amount of at least about ten percent of the administered gallium dose for at least about twenty-four hours following administration. In other embodiments of the present invention is provided a pharmaceutically acceptable formulation comprising a gallium compound, the gallium from which is retained within the lumen and tissues of the gastrointestinal tract in an amount of at least about twenty-five percent of the administered gallium dose for at least about twenty-four hours following administration.

In some of these embodiments, the formulation comprises a gallium compound selected from the group consisting of gallium nitrate, gallium sulfate, gallium citrate, gallium chloride, gallium complexes of 3-hydroxy-4-pyrones including gallium maltolate, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium (III)-gallium(III) tetrachloride, gallium pyridoxal isonicotinoyl hydrazone, gallium complexes of kenpaullone and its derivatives.

In other of these embodiments, the formulation comprises a gallium compound selected from the group consisting of gallium sulfate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, and gallium complexed with olsalazine.

In other of these embodiments, the formulation comprises a gallium compound selected from the group consisting of gallium hydroxide, gallium oxide hydroxide, gallium oxide, and gallium phosphate.

In other of these embodiments, the formulation comprises a gallium compound selected from the group consisting of gallium salicylate, gallium salicylate ethanol, gallium complexed with 5-aminosalicylic acid, gallium complexed with sulfasalazine, gallium complexed with balsalazide, and gallium complexed with olsalazine.

In other of these embodiments, the formulation comprises a gallium compound selected from the group consisting of gallium maltolate and gallium 8-quinolinolate. In other embodiments, the formulation comprises gallium maltolate.

In some of these embodiments, the formulation is a solid. In other embodiments, the formulation in the form of a tablet or a capsule. In other embodiments, the formulation is a liquid. In other embodiments, the formulation is in the form of a suppository.

### DETAILED DESCRIPTION OF THE INVENTION

Before the present formulations, methods, and drug delivery systems of the invention are disclosed and described, it is to be understood that this invention is not limited to specific formulations, i.e., specific carrier materials or the like, to specific dosage regimens, or to specific drug delivery systems, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a gallium compound” includes mixtures of such compounds; reference to “a carrier” includes mixtures of two or more carriers; and the like.

The term “inflammatory bowel disease” as used herein has its usual medical meaning, and refers to diseases including, but not limited to, Crohn's disease, ulcerative colitis, Johne's disease, Behçet's syndrome, collagenous colitis, diversion colitis, indeterminate colitis, infective colitis, ischaemic colitis, lymphocytic colitis, and closely related diseases and disorders of the gastrointestinal tract.

The terms “active agent,” “drug,” and “pharmaceutical agent” are used interchangeably herein to refer to a chemical material or compound which, when administered to an organism (e.g., human or animal, generally human) induces a desired pharmacologic effect, such as a reduction of inflammation.

The term “local delivery” as used herein refers to administration to spatially restricted portions of the body. For example, local delivery may include administration to portions of the gastrointestinal tract, as well as to other tissues and organs.

The term “individual” is meant to include an individual organism, preferably a vertebrate, more preferably a mammal, preferably a human. In some embodiments, the individual is human, including adults, children and premature infants. In some variations, the primate is a non-human primate such as a chimpanzee, another species of ape, or a monkey. In some embodiments, the mammal is a farm animal such as a cow, a horse, a sheep, a goat, or a pig. In other variations the mammal is a companion animal such as a rabbit, a dog, or a cat; a laboratory animal such as a member of the rodent family, which includes rats, mice, and guinea pigs; and the like. In some embodiments, the individual is a non-mammal. Examples of non-mammals include, but are not limited to, birds, reptiles, amphibians, and the like. The term “individual” does not denote a particular age or sex.

In some variations, the individual has been identified as having one or more of the conditions described herein. Identification of the conditions as described herein by a skilled physician is routine in the art and may also be suspected by the individual or others.

In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle, or habits.

As used herein, the term “pharmaceutically acceptable carrier,” and cognates thereof, refers to adjuvants, binders, diluents, etc. known to the skilled artisan that are suitable for administration to an individual (e.g., a mammal or non-mammal). Combinations of two or more carriers are also contemplated in the present invention. The pharmaceutically acceptable carrier(s) and any additional components, as described herein, should be compatible for use in the intended route of administration (e.g., oral, parenteral) for a particular dosage form. Such suitability will be easily recognized by the skilled artisan, particularly in view of the teaching provided herein.

As used herein, the terms “to treat” and “treatment” are defined as administering one or more of the compounds discussed herein, with or without additional therapeutic agents, in order to reduce or eliminate either the disease or one or more symptoms of the disease, or to retard the progression of the disease or of one or more symptoms of the disease, or to reduce the severity of the disease or of one or more symptoms of the disease, or to suppress the clinical manifestation of the disease, or to suppress the manifestation of adverse symptoms of the disease. Treatment may occur before or after adverse symptoms of the disease are manifest in an individual. Thus, for example, “treatment” of Crohn's disease, as the term “treatment” is used herein, encompasses both prevention of Crohn's disease in a predisposed individual and treatment of Crohn's disease in an individual who has such a disease. Treatment may be partial, substantially total, or total. As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition. For example, a partial or complete cure of inflammatory bowel disease may be indicated by a clinical improvement of inflammatory bowel disease, such as a decrease in abdominal pain and diarrhea.

The expression “an individual in need thereof” as used herein refers either to an individual who has been diagnosed with or previously treated for the condition to be treated, or to an individual who is at risk for a condition (e.g., a family history of the condition, life-style factors indicative of risk for the condition, etc.).

Unless defined otherwise or clearly indicated by context, all technical and scientific terms used herein have the same meaning as commonly understood by one of ordinary skill in the art to which this invention belongs.

This invention involves the use of pharmaceutically acceptable gallium compounds to treat or prevent inflammatory bowel disease and closely related disorders. Inflammatory bowel disease includes, without limitation, Behçet's syndrome, collagenous colitis, diversion colitis, indeterminate colitis, infective colitis, ischaemic colitis, and lymphocytic colitis. Closely related disorders comprise other inflammatory diseases and disorders of the gastrointestinal tract, including inflammation associated with infection (bacterial, viral, protozoan, parasitic, fungal, helminthic, or other), allergic or other sensitivity reactions, cancer, autoimmune disease, rheumatic disease, trauma, radiation, drugs, chemicals, or burns.

Gallium compounds usable in this invention include, without limitation, gallium nitrate, gallium sulfate, gallium citrate, gallium chloride, gallium complexes of 3-hydroxy-4-pyrones including gallium maltolate, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium(III)-gallium(III) tetrachloride, gallium pyridoxal isonicotinoyl hydrazone, gallium complexes of kenpaullone and its derivatives, and any other pharmaceutically acceptable gallium salts, organic salts, inorganic compounds, chelates, coordination compounds, and organometallic compounds. In some embodiments of the invention, gallium maltolate, tris(3-hydroxy-2-methyl-4H-pyran-4-onato)gallium, is used; this compound is described, for example, in U.S. Pat. No. 5,981,518, the content of which is hereby incorporated by reference.

This invention embraces pharmaceutical formulations suitable for the administration of gallium, and devices and methods for using such formulations to treat inflammatory bowel disease. Formulations of the pharmaceutical compositions appropriate for administration by any medically acceptable means are included in the invention. The pharmaceutical formulations may comprise a pharmaceutically acceptable carrier appropriate to the means of administration and a pharmaceutically acceptable gallium compound.

The gallium compounds described herein can be in formulations (including pharmaceutical compositions) with additives such as excipients (e.g., one or more excipients), antioxidants (e.g., one or more antioxidants), stabilizers (e.g., one or more stabilizers), preservatives (e.g., one or more preservatives), pH adjusting and/or buffering agents (e.g., one or more pH adjusting and/or buffering agents), tonicity adjusting agents (e.g., one or more tonicity adjusting agents), thickening agents (e.g., one or more thickening agents), suspending agents (e.g., one or more suspending agents), binding agents (e.g., one or more binding agents), viscosity-increasing agents (e.g., one or more viscosity-increasing agents), and the like, provided that the additional components are pharmaceutically acceptable for the particular condition to be treated. In some embodiments, the formulation may include combinations of two or more of the additional components as described herein (e.g., 2, 3, 4, 5, 6, 7, 8, or more additional components). In some embodiments, the additives include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-beta-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

The formulations may vary according to the condition to be treated, the amount of gallium compound to be administered, the condition of the individual, and other variables that will be readily apparent to one of ordinary skill in the art in view of the teachings provided herein.

The formulations comprising one or more compounds described herein may be administered in conjunction with one or more of the other pharmaceutical agents as described herein and as known in the art, including one or more additional pharmaceutical agents to further reduce the occurrence and/or severity of symptoms and/or clinical manifestations thereof, as well as pharmaceutical agents that treat or prevent the underlying conditions, or in conjunction with (e.g., prior to, concurrently with, or after) additional treatment modalities. The formulations as described herein may be administered before, concurrently with, or after the administration of one or more of the other pharmaceutical agents described herein. The compounds described herein may also be administered in conjunction with (e.g., prior to, concurrently with, or after) agents to alleviate the symptoms associated with either the condition or the treatment regimen.

In some embodiments, the other pharmaceutical agent(s) may be an immunosuppressant (e.g., azathioprine, methotrexate, 6-mercaptopurine, or mesalamine). In some embodiments, the other pharmaceutical agent(s) may be a steroid (e.g., prednisone). In some embodiments, the other pharmaceutical agent(s) may be a biological therapy (e.g., infliximab). Combinations of two or more of the foregoing may also be formulated, as can be determined by the skilled artisan in view of the teaching provide herein.

In some embodiments, the gallium compounds described herein and/or formulations described herein may be used in conjunction with (e.g., prior to, concurrently with, or after) surgical treatments (e.g., bowel resection, strictureplasty, and temporary or permanent colostomy or ileostomy).

The formulations described herein will generally be used in an amount effective to achieve the intended result, for example in an amount effective to treat or prevent the particular condition being treated or prevented. The formulations may be administered therapeutically to achieve therapeutic benefit. By therapeutic benefit is meant eradication or amelioration of the underlying condition being treated and/or eradication or amelioration of one or more of the symptoms associated with the underlying condition such that the individual reports an improvement in feeling or condition, notwithstanding that the individual may still be afflicted with the underlying condition. Therapeutic benefit also includes halting or slowing the progression of the condition, regardless of whether improvement is realized.

The amount of the formulation administered in order to administer an effective amount will depend upon a variety of factors, including, for example, the particular condition being treated; the frequency of administration; the particular formulation being administered; the severity of the condition being treated; the age, weight, and general health of the individual; the adverse effects experienced by the individual being treated; etc. Determination of an effective dosage is within the capabilities of those skilled in the art, particularly in view of the teachings provided herein. Dosages may also be estimated using in vivo animal models.

Administration of the compounds described herein may be by any medically acceptable means, including, but not limited to oral, subcutaneous, intramuscular, intravenous, intra-arterial, sublingual, buccal, rectal, peritoneal, nasal, transdermal, transmucosal, vaginal, transurethral, iontophoretic, and by inhalation. The compounds may be administered enterally (e.g., orally or rectally) or parenterally (e.g., by subcutaneous, intravenous, intramuscular, intrasternal, or peritoneal injection or infusion techniques) in dosage formulations containing conventional pharmaceutically acceptable carriers, adjuvants, and vehicles as desired. The compounds may be delivered locally, as to portions of the gastrointestinal tract. The route of administration may vary according to the condition to be treated. Additional methods of administration are known in the art.

The gallium compounds of the present invention may be mixed with pharmaceutically acceptable carriers, adjuvants, and vehicles appropriate for the desired route of administration. The compounds can be administered in solid form or in liquid form. The compounds can be administered in the form of tablets, pills, powder mixtures, capsules, granules, injectables, creams, solutions, suppositories, enemas, colonic irrigations, emulsions, dispersions, food premixes, animal feed, and in other suitable forms.

The gallium compounds of the invention may also be formulated using liposomes. Such formulations may be particularly advantageous for sustained release or delayed release. As is known in the art, liposomes are generally derived from phospholipids or other lipid substances. Liposomes are formed by mono- or multilamellar hydrated liquid crystals that are dispersed in an aqueous medium. Any nontoxic, physiologically acceptable and metabolizable lipid capable of forming liposomes can be used. The present formulations in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such formulations may also comprise adjuvants, such as wetting agents, emulsifying and suspending agents, cyclodextrins, and sweetening, flavoring, and perfuming agents.

The frequency and duration of administration of the formulation will depend on the condition being treated, the condition of the individual, and the like. The formulation may be administered to the individual one or more times, for example, 2, 3, 4, 5, 10, 15, 20, 50, 75, 100, or more times. The formulation may be administered to the individual, for example, once a day, two times a day, three times a day, or more than three times a day. The formulation may also be administered to the individual, for example, less than once a day, for example, every other day, every third day, every week, or less frequently. The formulation may be administered over a period of days, weeks, months, years, or chronically, such as life-long administration.

The amount of active agent that may be combined with the carrier materials to produce a single dosage form will vary depending upon the individual to which the active ingredient is administered and the particular mode of administration. It will be understood, however, that the specific dose level for any particular individual will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount for a given situation can be readily determined by routine experimentation and is within the skill and judgment of the ordinary clinician.

When additional active agents are used in combination with the compounds of the present invention, the additional active agents may generally be employed in therapeutic amounts as indicated in the Physicians' Desk Reference (PDR) 62nd Edition (2008), which is incorporated herein by reference, or such therapeutically useful amounts as would be known to one of ordinary skill in the art.

The compounds of the invention and the other therapeutically active agents can be administered at the recommended maximum clinical dosage or at lower doses. Dosage levels of the active compounds in the formulations of the invention may be varied so as to obtain a desired therapeutic response depending on the route of administration, severity of the disease and the response of the individual. When administered in combination with other pharmaceutical agents, the pharmaceutical agents can be formulated as separate formulations that are given at the same time or different times, or the pharmaceutical agents can be given as a single formulation.

In some embodiments, gallium compounds for oral administration include, for example, gallium maltolate, gallium 8-quinolinolate, gallium citrate, gallium nitrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, and gallium complexed with olsalazine. In some embodiments, gallium maltolate is administered orally.

In some embodiments, compounds for subcutaneous or intravenous administration include, but are not limited to, gallium nitrate, gallium citrate, and gallium 8-quinolinolate. In some embodiments, citrate-buffered gallium nitrate is administered subcutaneously or intravenously.

In some embodiments, gallium compounds for rectal administration via, for example, suppository or retention enema, include, but are not limited to, gallium maltolate, gallium 8-quinolinolate, gallium nitrate, gallium citrate, gallium phosphate, gallium salicylate, gallium salicylate ethanol, gallium complexed with 5-aminosalicylic acid, gallium complexed with sulfasalazine, gallium complexed with balsalazide, and gallium complexed with olsalazine. In some embodiments, gallium maltolate is administered rectally.

In some embodiments, the gallium compound or formulation is administered in an amount effective to treat the inflammatory bowel disease. Such amounts, for primarily systemic administration (that is, for distribution throughout the body via the blood and other body fluids), may result in plasma gallium concentrations of, for example, about 1 to 10,000 ng/mL, or about 100 to 5,000 ng/mL, or about 500 to 2,000 ng/mL. When the gallium formulation is administered for primarily local delivery within the gastrointestinal tract, such that the primary site of activity is a portion of the gastrointestinal tract and high absorption of gallium into the bloodstream is not sought, then plasma gallium concentrations may be lower.

In some embodiments, gallium compounds for primarily systemic oral administration include, for example, gallium maltolate, gallium 8-quinolinolate, gallium citrate, gallium nitrate, gallium salicylate, gallium salicylate ethanol, gallium complexed with 5-aminosalicylic acid, gallium complexed with sulfasalazine, gallium complexed with balsalazide, and gallium complexed with olsalazine. In some embodiments, gallium maltolate is used for primarily systemic oral administration. Although orally administered formulations comprising these compounds are thought to act primarily systemically, local activity of the compounds on the gastrointestinal tract as they pass through the gastrointestinal tract is also likely, and the invention is not restricted to any particular hypotheses regarding the means of activity.

In some embodiments, gallium compounds for primarily systemic parenteral administration include, for example, gallium nitrate, gallium citrate, and gallium 8-quinolinolate. In some embodiments, citrate-buffered gallium nitrate is used for primarily systemic parenteral administration. Although parentally administered formulations comprising these compounds are thought to act primarily systemically, local activity of the compounds on the gastrointestinal tract as they are excreted through the gastrointestinal tract is also possible, and the invention is not restricted to any particular hypotheses regarding the means of activity.

In some embodiments of the present invention is provided a pharmaceutically acceptable formulation for primarily local delivery within the gastrointestinal tract. In some such embodiments is presented a pharmaceutically acceptable formulation comprising a gallium compound, the gallium from which is retained within the lumen and tissues of the gastrointestinal tract in an amount of at least about ten percent of the administered gallium dose for at least about twelve hours following administration. In other such embodiments of the present invention is provided a pharmaceutically acceptable formulation comprising a gallium compound, the gallium from which is retained within the lumen and tissues of the gastrointestinal tract in an amount of at least about twenty-five percent of the administered gallium dose for at least about twelve hours following administration. In other such embodiments of the present invention is provided a pharmaceutically acceptable formulation comprising a gallium compound, the gallium from which is retained within the lumen and tissues of the gastrointestinal tract in an amount of at least about ten percent of the administered gallium dose for at least about twenty-four hours following administration. In other such embodiments of the present invention is provided a pharmaceutically acceptable formulation comprising a gallium compound, the gallium from which is retained within the lumen and tissues of the gastrointestinal tract in an amount of at least about twenty-five percent of the administered gallium dose for at least about twenty-four hours following administration.

In some embodiments, gallium compounds for primarily local delivery within the gastrointestinal tract, administered orally, include, for example, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, and gallium complexed with olsalazine. In some embodiments, gallium phosphate is used for primarily local delivery within the gastrointestinal tract, administered orally. Although orally administered formulations comprising these compounds are thought to act primarily locally within the gastrointestinal tract, systemic activity may occur, and the invention is not restricted to any particular hypotheses regarding the means of activity.

In some embodiments of primarily systemic oral administration, gallium maltolate is administered orally at a dose of, for example, about 50 to 5,000 mg/day, or about 200 to 3,000 mg/day, or about 300 to 2,000 mg/day, together with a pharmaceutically acceptable carrier. In some embodiments, the dose may be administered in a single dose once per day or in divided doses two or more times per day.

In some embodiments of parenteral systemic administration, citrate-buffered gallium nitrate is administered, for example, intravenously in a pharmaceutically acceptable intravenous liquid formulation, such as a slow infusion. The gallium nitrate may be administered, for example, at a Ga(NO₃)₃ dose of about 10 to 1,000 mg/m²/day, or about 100 to 500 mg/m²/day, as a continuous intravenous infusion for about 1 to 10 days, or about 3 to 7 days. In some embodiments, this dose may be repeated about every 1 to 12 weeks, or about every 2 to 4 weeks.

In some embodiments of administration for primarily local delivery within the gastrointestinal tract, gallium phosphate is administered orally at a dose of, for example, about 10 to 5,000 mg/day, or about 100 to 2,500 mg/day, or about 250 to 2,000 mg/day, together with a pharmaceutically acceptable carrier. The dose may be administered in a single dose once per day, or in divided doses two or more times per day.

In some embodiments of a formulation for primarily local delivery within the gastrointestinal tract, a tablet is prepared by conventional means that contains, for example, about 500 mg of gallium phosphate, about 2.5 mg magnesium stearate, about 40 mg microcrystalline cellulose, and about 2 mg carboxymethylcellulose sodium.

In some embodiments of rectal administration by suppository, gallium maltolate is administered in a daily dose of, for example, about 1 to 2,000 mg/day, or about 10 to 1,000 mg/day, or about 25 to 250 mg/day.

Suppositories may be prepared by conventional means, using suitable non-irritating excipients or carriers such as cocoa butter, polyethylene glycol, or a suppository wax, which are solid at room temperature but are liquid or partially liquid at body temperature; they therefore melt in the rectum, releasing the active compound. Rectal administration may be particularly desirable when the rectum is affected by inflammatory bowel disease, as in ulcerative colitis. Rectal administration may also be particularly desirable in cases where the individual is unable to receive, or cannot tolerate, orally administered medication.

In some embodiments of a suppository formulation, a mixture containing about 2 weight percent gallium maltolate, about 74 weight percent polyethylene glycol 1000, and about 24 weight percent polyethylene glycol 4000 is prepared. This mixture may be melted and mixed on a steam bath and poured into molds each containing, for example, about 2.5 g total weight of the formulation. Suppositories are thus produced that each weigh, for example, about 2.5 g and contain about 50 mg of gallium maltolate.

The practice of the present invention will employ, unless otherwise indicated, conventional techniques of drug formulation that are within the skill of the art. Such techniques are fully explained in the literature. See, for example, Remington: The Science and Practice of Pharmacy (2000), cited supra, as well as Goodman & Gilman's The Pharmacological Basis of Therapeutics, 9th Ed. (New York: McGraw-Hill, 1996) and Ansel et al., Pharmaceutical Dosage Forms and Drug Delivery Systems, 6^{th} Ed. (Media, Pa.: Williams & Wilkins, 1995).

All patents, patent documents, and publications cited herein are hereby incorporated by reference in their entirety for their disclosure concerning any pertinent information not explicitly included herein.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains.

1. A method for treating inflammatory bowel disease in an individual in need thereof, comprising administering to the individual a therapeutically effective amount of a pharmaceutically acceptable gallium compound.

2. The method of claim 1, wherein the inflammatory bowel disease is Crohn's disease, ulcerative colitis, Johne's disease, Behçet's syndrome, collagenous colitis, diversion colitis, indeterminate colitis, infective colitis, ischaemic colitis, or lymphocytic colitis.

3. The method of claim 2, wherein the inflammatory bowel disease is Crohn's disease, ulcerative colitis, or Johne's disease.

4-6. (canceled)

7. The method of claim 1, wherein the gallium compound is selected from the group consisting of gallium nitrate, gallium sulfate, gallium citrate, gallium chloride, gallium complexed with a 3-hydroxy-4-pyrone, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium(III)-gallium(III) tetrachloride, gallium pyridoxal isonicotinoyl hydrazone, gallium complexed with kenpaullone, and gallium complexed with a kenpaullone derivative.

8. (canceled)

9. The method of claim 7, wherein the gallium compound is gallium maltolate.

10. The method of claim 1, wherein the gallium compound is administered orally.

11-13. (canceled)

14. The method of claim 10, wherein the gallium compound is selected from the group consisting of gallium nitrate, gallium citrate, gallium chloride, gallium maltolate, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, and gallium 8-quinolinolate.

15. The method of claim 14, wherein the gallium compound is gallium maltolate.

16. The method of claim 1, wherein the gallium compound is administered rectally.

17. (canceled)

18. The method of claim 16, wherein the formulation is in the form of a suppository.

19. The method of claim 16, wherein the gallium compound is selected from the group consisting of gallium nitrate, gallium citrate, gallium chloride, gallium complexes of 3-hydroxy-4-pyrones including gallium maltolate, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium (III)-gallium(III) tetrachloride, gallium pyridoxal isonicotinoyl hydrazone, gallium complexes of kenpaullone and its derivatives.

20. The method of claim 19, wherein the gallium compound is gallium maltolate.

21. A pharmaceutically acceptable formulation comprising a gallium compound, the gallium from which is retained within the lumen and tissues of the gastrointestinal tract in an amount of at least about ten percent of the administered gallium dose for at least about twelve hours following administration.

22. (canceled)

23. The formulation of claim 21, the gallium from which is retained within the lumen and tissues of the gastrointestinal tract in an amount of at least about ten percent of the administered gallium dose for at least about twenty-four hours following administration.

24. (canceled)

25. The formulation of claim 21, wherein the gallium compound is selected from the group consisting of gallium nitrate, gallium sulfate, gallium citrate, gallium chloride, gallium complexes of 3-hydroxy-4-pyrones including gallium maltolate, gallium tartrate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, gallium complexed with olsalazine, gallium succinate, gallium gluconate, gallium palmitate, gallium 8-quinolinolate, gallium porphyrins including gallium(III) protoporphyrin IX, gallium transferrin, bis(2-acetylpyridine 4N-dimethylthiosemicarbazone)gallium (III)-gallium(III) tetrachloride, gallium pyridoxal isonicotinoyl hydrazone, gallium complexes of kenpaullone and its derivatives.

26. The formulation of claim 25, wherein the gallium compound is selected from the group consisting of gallium sulfate, gallium hydroxide, gallium oxide hydroxide, gallium oxide, gallium phosphate, gallium salicylate, gallium salicylate ethanol, gallium complexed with 5-aminosalicylic acid, gallium complexed with sulfasalazine, gallium complexed with balsalazide, and gallium complexed with olsalazine.

27. (canceled)

28. The formulation of claim 26, wherein the gallium compound is selected from the group consisting of gallium salicylate, gallium salicylate ethanol, gallium complexed with 5-aminosalicylic acid, gallium complexed with sulfasalazine, gallium complexed with balsalazide, and gallium complexed with olsalazine.

29. (canceled)

30. The formulation of claim 25, wherein the gallium compound is gallium maltolate.

31. The formulation of claim 25, in the form of a solid.

32-33. (canceled)

34. The formulation of claim 21, in the form of a suppository.
